(19) United States
(12) Patent Application Publication
(10) Pub. No.: US 2023/0210866 A1
(43) Pub. Date: Jul. 6, 2023

(54) COMPOSITION COMPRISING DILTIAZEM FOR TREATING A VIRAL INFECTION CAUSED BY SARS-COV-2 VIRUSES

(71) Applicants: UNIVERSITE CLAUDE BERNARD LYON 1, VILLEURBANNE (FR); INSERM (Institut National de la Sante et de la Recherche Medicale), PARIS (FR); CENTRE NATIONAL DE LA RECHERCHE SCIENTIFIQUE (CNRS), PARIS (FR); ECOLE NORMALE SUPERIEURE DE LYON, LYON (FR); SIGNIA THERAPEUTICS, LYON (FR)

(72) Inventors: Manuel ROSA-CALATRAVA, LYON (FR); Olivier TERRIER, LYON (FR); Mario Andres PIZZORNO, LYON (FR); Aurelien TRAVERSIER, VENISSIEUX (FR); Blandine PADEY, LYON (FR); Thomas JULIEN, VILLETTE D’ANTHON (FR)

(21) Appl. No.: 17/905,562

(22) PCT Filed: Mar. 10, 2021
(86) PCT No.: PCT/FR2021/050406

§ 371 Date: Sep. 2, 2022

(30) Foreign Application Priority Data
Mar. 10, 2020 (FR) FR2002351

(51) Int. Cl. A61K 31/554; A61P 31/14; A61K 31/7076; A61K 45/06

(52) U.S. Cl. CPC A61K 31/554; A61K 31/7076; A61K 45/06; A61P 31/14

(57) ABSTRACT

The present invention relates to a pharmaceutical composition comprising Diltiazem in a suitable pharmaceutical carrier for its therapeutic use in the prevention and/or treatment of a viral infection caused by the SARS-CoV-2 virus, referred to as COVID-19 disease.

### FIELD OF THE INVENTION

The present invention relates to a novel therapeutic use of known therapeutic compounds, alone or in combination with other active ingredients, for treating viral infection by the severe acute respiratory syndrome coronavirus SARS-CoV-2, otherwise referred to as coronavirus 2019 disease (COVID-19).

### PRIOR ART

Human acute respiratory infections (ARI) are one of the main causes of consultations, hospitalisations and deaths in the world, being in particular the primary cause of death in young children, with nearly 2 million deaths per year. Viruses occupy a prominent place among the etiological agents responsible for acute respiratory infections. More particularly, they are found in the majority of cases of paediatric pneumonia and are a predisposing factor for bacterial pneumonia in adults.

Coronaviruses are enveloped viruses, having a capsid exhibiting a helical symmetry. They have a single positive-strand RNA genome and are capable of infecting the cells of birds and mammals

Coronavirus infections can cause respiratory diseases associated with symptoms similar to the common cold (caused in particular by the hCoV and OC43 viruses), bronchiolitis (caused by the NL63 virus) or even more severe diseases such as severe acute respiratory syndrome caused by SARS-CoV (Severe Acute Respiratory Syndrome Coronavirus) having generated an epidemic in 2003, and the Middle East respiratory syndrome due to MERS-CoV, having generated an epidemic in 2012.

Severe acute respiratory syndrome coronavirus 2, or SARS-CoV-2, is the coronavirus at the origin of the coronavirus epidemic of 2019-2020, generating the form of pneumonia named coronavirus disease 2019 (COVID-19). This epidemic was declared a “public health emergency of international concern” by the World Health Organisation (WHO) on 30 Jan. 2020. The first patient was reported in the city of Wuhan (Hubei province, People’s Republic of China). Up to 9 Mar. 2020, 111,321 cases had been confirmed worldwide, including 3892 deaths in 109 countries.

Related to the SARS-CoV virus, SARS-CoV-2 virus belongs to the species of Severe acute respiratory syndrome-related coronavirus, in the genus Betacoronavirus and the family Coronaviridae (Gorbalenya et al., 2020). The morphology of the virions is typical of that of coronaviruses, having, in particular, the halo spike proteins which give them their name “corona virus”.

Its genome, consisting of single-stranded RNA with 29,903 nucleotides, was sequenced for the first time on 5 Jan. 2020 by a team at Fudan University in Shanghai (China). Its genome is similar at 79.5% to that of SARS-CoV and at 96% to that of bat coronavirus, which suggests that the origin of the SARS-CoV-2 virus is zoonotic and is found in bats (Zhou et al., 2020).

The symptoms of infection by SARS-CoV-2 largely resemble those of seasonal flu: they include fever, tiredness, a dry cough, shortness of breath, breathing difficulties, pneumonia and kidney failure, and can lead to death of the patient in severe cases (Hui et al., 2020).

A calculation of lethality, based on the figures supplied to the WHO by various countries on confirmed infections and deaths, makes it possible to estimate the mortality rate at approximately 2% (data from February 2020). Nevertheless, this fatality rate remains uncertain due to the difficulty of estimating, in the field, the number of confirmed cases and the number of deaths directly attributable to SARS-CoV-2. Indeed, it should be noted that, according to the WHO, the majority of deceased patients had an immune system that was weakened due to other health problems, such as high blood pressure, diabetes or cardiovascular disease.

The incubation period of SARS-CoV-2 is estimated to be between 2 and 14 days, but the incubation period could be longer in some cases, up to 24 days.

No specific antiviral treatment or vaccine is currently available for the prevention and/or treatment of viral infection with SARS-CoV-2.

The majority of treatments currently received by patients infected with this virus essentially aim to alleviate the symptoms of fever, coughing and dyspnoea, in order to promote their spontaneous recovery.

Antiviral compounds, known for their therapeutic activity on other types of virus, are currently being tested in phase 2 and phase 3 clinical trials. They concern, in particular, cellular or viral protease inhibitor compounds, and nucleoside analogues. These different therapeutic compounds include, in particular, remdesivir, galidesivir, lopinavir, camostat mesilate and chloroquine.

### Remdesivir

Remdesivir is a broad-spectrum antiviral compound, acting as a nucleoside analogue, more particularly as an adenosine analogue. Available in the form of a lyophilisate, this compound is administered intravenously. It is metabolised into its active nucleotide analogue form (GS-441524): its presence misleads the viral polymerase and causes a reduction in viral RNA synthesis.

Remdesivir was developed by Gilead Science for the treatment of infections by the Ebola and Marburg viruses. It also has antiviral activity against other single-strand RNA viruses such as the Nipah, Hendra and Lassa viruses, respiratory syncytial virus (RSV) and SARS and MERS coronaviruses, in preclinical models (Lo et al., 2017).

In clinics, remdesivir has so far been studied for healthy volunteers and in patients infected with the Ebola virus. The treatments prescribed to some patients infected with SARS-CoV-2 have not provided information regarding the safety and/or efficacy in treating SARS-CoV-2 infection. In addition, given the limited clinical experience, the toxicity profile and therefore the safety profile of this compound is not currently characterised. Remdesivir has not yet received marketing authorisation.

In this context, Gilead has supplied remdesivir to China in order to evaluate it in a plurality of clinical tests including patients infected with SARS-CoV-2 (with or without symptoms). On 26 Feb. 2020, Gilead announced the performance of two phase-3 clinical trials to test this compound.

### Galidesivir

Galidesivir (BCX4430) is a nucleoside analogue, in particular of adenosine, developed by BioCryst Pharmaceuticals.

This antiviral compound was initially developed for use against the hepatitis-C virus, then subsequently against filoviruses such as the Ebola virus and Marburg virus. Galidesivir protects against Ebola virus disease and Marburg virus disease in rodents and monkeys, when it is administered up to 48 hours after exposure to the virus. Development for use in humans has been accelerated in order to fill the lack of available treatments for combating the epidemic of Ebola virus disease in West Africa.

This compound has a broad-spectrum antiviral activity against all other families of RNA virus, such as bunyaviruses, arenaviruses, paramyxoviruses, coronaviruses and flaviviruses.

### Molnupiravir

Molnupiravir (also designated by the abbreviations EIDD-2801 and MK-4482) is an antiviral compound which has the advantage of being in a form suitable for oral administration.

This compound is a pro-drug that is metabolised after ingestion into N4-hydroxycytidine, a nucleoside derivative which inhibits RNA viruses by introducing errors in RNA replication by the viral RNA-dependent RNA polymerase.

This compound has been tested for its anti-influenza activity. It also has some activity against coronaviruses such as SARS-CoV, MERS-CoV and SARS-CoV-2, respectively responsible for the so-called SRAS, MERS and COVID-19 diseases. (Painter et al., 2021); (Sheahan et al., 2020).

### Lopinavir

Iopinavir is a viral protease inhibitor, used as an antiviral against the human immunodeficiency virus (HIV). It is marketed in combination with ritonavir by Abbott Laboratories, substantially under the name Kaletra.

Iopinavir acts by inhibiting the production of functional proteins and enzymes by the new virions, which blocks the propagation of the virus.

In humans, lopinavir is rapidly broken down in the organism by the cytochrome P450 system. This is the reason why it is administered in combination with ritonavir. The function of this second drug, which is also a protease inhibitor, is to inhibit the monooxygenases of cytochrome P450, and thus to slow the breakdown of lopinavir by these enzymes. This makes it possible to substantially reduce the required dose and thus the number of tablets which need to be absorbed by the patient.

A study published in 2004 has shown that the iopinavir/ritonavir combination has a “substantial clinical benefit” for patients with SARS (Chu et al., 2004).

With regards to the treatment of SARS-CoV-2, the Wuhan Jinyintan Hospital, where the first 41 confirmed patients of the COVID-19 disease were treated, has begun a randomised control trial with this combination of anti-HIV drugs.

### Camostat Mesilate

Camostat mesilate (FOY-305) is a low molecular weight synthetic protease inhibitor. It is capable of inhibiting trypsin, prostasin, matriptase and plasma kallikrein. This compound is used in therapy for treating chronic inflammation of the pancreas. In addition, this compound attenuates the function of epithelial sodium channels of the respiratory tract and improves mucociliary clearance.

Camostat mesilate tablets are approved in Japan and distributed under the brand name FOIPAN®. They are used for the treatment of remission of acute symptoms of chronic pancreatitis and post-operative reflux oesophagitis.

Patent EP 2 435 064 proposes targeting the serine proteases HAT and TMPRSS2 for the treatment of viral infections, in particular by the influenza virus.

Recently, it has been shown that camostat mesilate inhibits transmembrane serine protease 2, TMPRSS2, of the SARS-CoV-2, an enzyme necessary for multiplication of the virus (Hoffmann et al., 2020).

### Chloroquine

Chloroquine, initially known for its antipaludic activity, has an antiviral action on SARS-CoV according to data obtained in vitro (Vincent et al., 2005).

Recently published studies have described an apparent efficacy of chloroquine phosphate in the treatment of the SARS-CoV-2 virus. (Wang et al., 2020; Gao et al., 2020)

Thus, a consensus of Chinese experts now recommends including chloroquine phosphate in the treatment of patients, with 500 mg twice per day for 10 days for patients diagnosed as mild, moderate and severe cases of disease, and without contraindication for chloroquine.

The clinical data are however still limited.

New antiviral compounds are being actively sought for the treatment and prevention of this emerging virus, SARS-CoV-2.

The new antiviral compounds identified in recent years include, in particular, diltiazem, which is an active compound which acts on the host cell of the virus rather than directly on the virus, thus enabling it to have a broad-spectrum action and to be able to treat various viral infections.

International application WO 87/07508 describes the use of diltiazem for treating viral infections linked to the cytomegalovirus or to herpes.

International application WO 2011/066657 describes the use of diltiazem for the treatment or prevention of viral infections such as oral herpes, genital herpes and zoster.

International application WO 2016/146836 describes the use of diltiazem for treating infections by the influenza virus.

The article of (Fujioka et al., 2018), teaches that diltiazem, known for its role as an inhibitor of the transport of Ca2+ ions, also has an inhibitory action on cellular infection by the influenza A virus.

International application WO 2019/224489 discloses, in detail, some of the biological effects of diltiazem, which induces the expression of genes coding for type III interferons in the cells of the respiratory epithelium and the intestinal epithelium. Diltiazem has thus been proposed for various therapeutic applications, in particular for the treatment of viral and bacterial respiratory infections in the respiratory and intestinal epithelia.

Like diltiazem, berberine is an active compound known to have an antiviral activity.

Berberine is an isoquinoline alkaloid produced by certain plants, in particular by the species Berberi, which is used a lot in the Asiatic pharmacopoeia.

Studies carried out in vitro and in a mouse model have shown that berberine acts on various cellular pathways, and has antibacterial, antifungal, antiviral, anti-inflammatory and metabolic properties, improving hyperglycaemia and type II diabetes, as well as hyperlipidemia. (Mohan et al., 2017)

Berberine is, in particular, active for combating pulmonary arterial hypertension (PAHT), a diffuse disease of pulmonary microvascular remodelling accompanied by a malign proliferation of smooth muscle cells of the pulmonary artery, which causes a persistent increase in the pulmonary arterial pressure leading to right ventricular hypertrophy (RVH).

Berberine also has excellent anti-inflammatory properties, reducing joint swelling, the infiltration of tissues and their destruction by inflammatory cells. Berberine regulates the polarisation of macrophages, reduces the phagocyte function of macrophages, reduces the content of M1 cytokines and increases the level of M2 cytokines (IL-10 and transforming growth factor-β1 or TGF-β1).

Berberine has a broad-spectrum antibacterial activity, in particular against infection by methicillin-resistant Staphylococcusaureus (MRSA). When it is applied in vitro and in combination with methoxyhydnocarpin, berberine inhibits the growth of Staphylococcusaureus.

Berberine has an antiviral activity on various viruses, in particular:
on the herpes virus HSV (Song et al. 2014),
on the influenza virus (Wu et al. 2011),
on the cytomegalovirus (HCMV): low (micromolar) concentrations of berberine inhibit the replication of various strains of HCMV, including clinical isolates and strains resistant to approved inhibitors of DNA polymerase, by interfering with the transactivation activity of the viral protein Immediate Early-2 (IE2) (Luganini et al., 2019),
on the hepatitis-C virus (HCV): berberine would inhibit the entry of HCV into the cells (Ting-Chun Hung et al., 2019),
on Zika virus (ZIKV): berberine and emodin have been shown to be particularly effective against the entry and replication of several viruses, triggering in particular a powerful virucidal effect on ZIKV (Batista et al. 2019).

With respect to coronavirus:
berberine may have an antiviral action against SARS-CoV (WO 2013/185126), although no experimental data is presented; and
berberine has an antiviral action against MERS-CoV (WO 2018/073549).

In the context of the present invention, a plurality of compounds have been selected and evaluated in a cellular test of viral infection and on a human respiratory epithelium model cultivated in vitro at the air-liquid interface. Some of these compounds, alone or in combination, have exhibited an antiviral effect on SARS-CoV-2 coronavirus, in an entirely unexpected manner.

The selected compounds have been previously described for other therapeutic applications. Surprisingly, it has now been shown that these compounds, alone or in combination, have an antiviral activity against SARS-CoV-2, and enable the treatment and/or prevention of infection by this coronavirus.

In particular, the combination therapies based on the combination of at least two antiviral compounds is particularly effective for the treatment and/or prevention of infection by coronavirus SARS-CoV-2.

### DISCLOSURE OF THE INVENTION

The present invention relates to a compound chosen from diltiazem, berberine and their combination, for the therapeutic use thereof in the treatment of viral infection by the SARS-CoV-2 virus (so-called COVID-19 disease).

In particular, the present invention relates to diltiazem, for the therapeutic use thereof in the prevention and/or treatment of the viral infection by the SARS-CoV-2 virus, referred to as COVID-19 disease.

Moreover, the present invention relates to a pharmaceutical composition comprising, in a suitable pharmaceutical carrier, at least one compound chosen from diltiazem and berberine, for the therapeutic use thereof in the treatment of the viral infection by the SARS-CoV-2 virus (so-called COVID-19 disease).

The present invention also relates to a pharmaceutical composition comprising diltiazem in a suitable pharmaceutical carrier, for the therapeutic use thereof in the prevention and/or treatment of the viral infection by the SARS-CoV-2 virus.

The present invention also relates to a composition for the use thereof as described above, comprising at least one other active ingredient chosen from:
a nucleoside analogue;
a viral protease inhibitor;
a transmembrane serine protease inhibitor;
chloroquine, and
any mixture of the above compounds.

Among the nucleoside analogues, the preferred compounds are remdesivir, galidesivir, molnupiravir and the combinations thereof.

Among the viral protease inhibitors, the preferred compound is lopinavir and, more preferably, lopinavir combined with ritonavir.

Among the transmembrane serine protease inhibitors, the preferred compound is camostat mesilate.

The present invention also relates to a combination product comprising at least one compound chosen from diltiazem and berberine, and at least one other active ingredient chosen from:
a nucleoside analogue;
a viral protease inhibitor;
a transmembrane serine protease inhibitor;
chloroquine, and
any mixture of the above compounds.
for the simultaneous, separated or sequential use thereof in the prevention and/or treatment of a viral infection by the virus SARS-CoV-2.

The present invention also relates to a combination product comprising diltiazem and at least one other active ingredient chosen from:
a nucleoside analogue;
a viral protease inhibitor;
a transmembrane serine protease inhibitor;
chloroquine, and
any mixture of the above compounds,
for the simultaneous, separated or sequential use thereof in the prevention and/or treatment of a viral infection by the SARS-CoV-2 virus.

Finally, the invention also relates to a pharmaceutical composition comprising, in a suitable pharmaceutical carrier, a combination of diltiazem with remdesivir; or a combination of diltiazem with molnupiravir.

### DESCRIPTION OF THE FIGURES

FIG. 1 shows the antiviral effects of berberine and remdesivir in monotherapy on Vero E6 cells in culture infected by the SARS-CoV-2 virus.

FIG. 1A: timing diagram of the experiment.

FIG. 1B: Curves of effect versus dose of berberine and remdesivir molecules in monotherapy and determination of the associated IC50 at 48 and 72 hours post-infection (hpi). Remdesivir and berberine have an antiviral effect at 48 hpi, with an IC50 determined at 0.98 and 17.47 µM, respectively. This antiviral effect is even more pronounced at 72 hpi with IC50 less than 0.72 and 5.60 µM for remdesivir and berberine, respectively.

FIG. 2 shows the antiviral effects of diltiazem/remdesivir and berberine/remdesivir combinations on Vero E6 cells in culture infected by the SARS-CoV-2 virus.

FIG. 2A: timing diagram of the experiment.

FIG. 2B: Curves of effect versus dose for diltiazem/remdesivir and berberine/remdesivir combinations, and determination of the associated IC50 at 48 and/or 72 hours post-infection (hpi). The combination of a treatment by remdesivir in the presence of a fixed concentration of diltiazem (11.5 µM), makes it possible to obtain an antiviral efficacy with an IC50 of 0.32 µM, at 48 hpi. In equivalent manner, the combination of a treatment by diltiazem in the presence of a fixed concentration of remdesivir (2.5 µM), makes it possible to obtain an antiviral efficacy with an IC50 of 0.55 µM, at 48 hpi. Furthermore, the combination of a treatment by remdesivir in the presence of a fixed concentration of berberine (12.5 µM), makes it possible to have an antiviral efficacy with an IC50 of 0.65 µM, at 48 hpi. At a more advanced stage of the infection (72 hpi) the combination of a treatment by remdesivir in the presence of a fixed concentration of diltiazem (11.5 µM), makes it possible to obtain an antiviral efficacy with an IC50 of 0.35 µM.

FIG. 3 shows certain results presented in FIGS. 1B and 2B in visual manner.

FIG. 3A: timing diagram of the experiment.

FIG. 3B: Observation by photon microscopy of the antiviral effect of remdesivir and diltiazem in monotherapy and in combination on a carpet of Vero E6 cells in culture, and infected by SARS-CoV-2. Under the same experimental conditions as before (FIG. 2), the combination of remdesivir (0.625 µM) and diltiazem (11.5 µM) very visibly reduces the cytopathic effects of the infection (rounded cells detached from the carpet) compared to the untreated control, and monotherapy treatments.

FIG. 4: Timing diagram of the experiments of example 3.

FIG. 5. Timing diagram of the experiment of example 4. The A549-ACE2 cells are inoculated then infected with a viral strain of SARS-CoV-2, before being treated 1 hour post-infection with diltiazem or remdesivir.

FIG. 6. (A) A549-ACE2 infected at a MOI concentration of 10⁻¹; (B) A549-ACE2 cells infected at an MOI concentration of 10⁻².

The cells were not treated (black curves) or treated with remdesivir 5 µM (grey squares) or with diltiazem 45 µM (grey triangles). The results are expressed in percentage of viral titre measured in the supernatant, compared to the viral titre measured in the wells of the untreated cells, as a function of the time after viral infection.

FIG. 7. (A). Measurement of the IC50 of diltiazem on A549-ACE2 cells infected by a viral strain of SARS-CoV-2. The viral titre expressed in percentage of the viral titre measured in the supernatant, compared to the viral titre measured in the wells of untreated cells, as a function of the concentration of diltiazem used. (B). Measurement of the CC50 of diltiazem on A549-ACE2 cells after 72 hours of treatment. The viability of the cells is expressed as a percentage of the viability measured for the untreated cells. The results are presented as a function of the concentration of diltiazem used.

### DETAILED DESCRIPTION OF THE INVENTION

The present invention relates to diltiazem for its therapeutic use in the prevention and/or treatment of the viral infection by the SARS-CoV-2 virus, referred to as COVID-19 disease.

The present invention also relates to a pharmaceutical composition comprising, in a suitable pharmaceutical carrier, at least one compound chosen from diltiazem and berberine, for its therapeutic use in the treatment of viral infection by the SARS-CoV-2 virus (COVID-19).

The present invention also relates to a pharmaceutical composition comprising diltiazem in a suitable pharmaceutical carrier, for its therapeutic use in the prevention and/or treatment of the viral infection by the SARS-CoV-2 virus.

“Viral infection by SARS-CoV-2 virus” means the fact that a human or animal organism has cells having been infected by SARS-CoV-2 coronavirus, also referred to by the name COVID-19.

Within the meaning of the invention, the expression “SARS-CoV-2 virus” means, on the one hand, the coronavirus identified for the first time at Wuhan in China and sequenced at the start of 2020 by a team from the Fudan University in Shanghai (Zhou et al., 2020); and on the other hand, includes all the variants related to this first identified viral strain, which appeared later and, in particular, the following SARS-CoV-2 variant strains:
i. the “Wuhan-like” strain used by the inventors, described in the examples;
ii. the strain hCoV-19/France/ARA-104350/2020 (GISAID ID: EPI_ISL_683350) of the B.1 lineage (this strain has at least the D614G mutation in its spike protein; it is now considered as the wild strain circulating in Europe, compared with the variants cited below);
iii. a viral strain referred to as the “English variant” hCoV-19/France/ARA-SC2118/2020 (1D GISAID: EPI_ISL_900512) of the lineage B.1.1.7;
iv. a South African strain (501Y.V2.HV001) of the lineage B.1.3.5.1 and
v. a Brazilian strain of the lineage B.1.1.28.

A viral infection is generally diagnosed by a health professional on the basis of observation of the symptoms of the infected patient. Complementary biological examinations may be necessary in order to confirm the diagnosis: analyses of blood and/or expectorations and/or bronchoalveolar fluid.

In particular, the infection can be established by carrying out a detection by molecular biology and/or a viral titration based on respiratory samples, or by assaying specific antibodies of SARS-CoV-2 circulating in the blood.

The detection of this specific virus in infected individuals is performed by conventional diagnostic methods, in particular from molecular biology (PCR) which can establish that it indeed concerns this SARS-CoV-2 virus, which methods are well known to a person skilled in the art.

The term “treatment” designates the fact of combating the infection by coronavirus SARS-CoV-2 in a human or animal organism. Through the administration of at least one composition according to the invention, the level of viral infection (infectious titre) in the organism will be reduced, and preferably the virus will disappear completely from the organism within a shorter time than that expected for a recovery without treatment.

The term “treatment” also designates the fact of alleviating the symptoms associated with the viral infection (respiratory syndrome, kidney failure, fever, etc.).

Certain compositions according to the invention are also intended for use in the prevention of an infection by SARS-CoV-2.

Hence, the present invention relates to:
a pharmaceutical composition comprising diltiazem in a suitable pharmaceutical carrier, for the therapeutic thereof in the treatment of the viral infection by the SARS-CoV-2 virus.
a pharmaceutical composition comprising berberine in a suitable pharmaceutical carrier, for the therapeutic thereof in the treatment of the viral infection by the SARS-CoV-2 virus; and
a pharmaceutical composition comprising diltiazem in a suitable pharmaceutical carrier, for the use thereof in the prevention of viral infection by the SARS-CoV-2 virus.

Within the meaning of the invention, the term “prevention” designates the fact of preventing, or at least reducing the probability of appearance of, an infection in a human or animal organism by SARS-CoV-2. Through the administration of at least one composition according to the invention, the human or animal cells of said organism become less permissive of the infection, and are thus more likely not to be infected by said coronavirus, or to develop less severe symptoms during the infection by said coronavirus.

The compositions according to the invention can be of pharmaceutical type, intended to be administered to a human being, or of veterinary type, intended to be administered to nonhuman animals. Concerning animals, it is expected that veterinary compositions for their use in the prevention and/or treatment of infection by coronavirus SARS-CoV-2 are intended to be administered to animals infected by this coronavirus.

According to the invention, the term “suitable pharmaceutical carrier” designates pharmaceutical carriers or excipients, which are compounds not having their own action on the infection considered here. These carriers or excipients are pharmaceutically acceptable, which means that they can be administered to an individual or to an animal without generating significant deleterious effects.

The expression “at least one compound chosen from diltiazem and berberine” means that the pharmaceutical composition comprises either diltiazem or berberine or a combination of the two.

According to a first aspect, the pharmaceutical composition for use thereof according to the invention comprises at least an effective quantity of diltiazem. This pharmaceutical composition is intended for therapeutic and/or preventive use against infection by the SARS-CoV-2 virus.

According to a second aspect, the pharmaceutical composition for use thereof according to the invention comprises at least an effective quantity of berberine.

According to a third aspect, the pharmaceutical composition for use thereof according to the invention comprises at least an effective quantity of diltiazem and an effective quantity of berberine.

The term “effective quantity” means, within the meaning of the invention, a sufficient quantity of active compound to inhibit the proliferation and/or replication of the coronavirus, and/or the development of the viral infection within the organism. This inhibition can be quantified, for example by measuring the viral titre, as this is exhibited in the examples of the present application.

Thus, according to a particular aspect of the invention, the pharmaceutical composition for the use thereof as described above comprises a combination of diltiazem and berberine.

The term “combination” means, within the meaning of the invention, a composition comprising at least two distinct active compounds, both compounds having an antiviral action.

This combination comprises either the same quantity, by weight, of each antiviral compound, i.e. a combination of 50% diltiazem and 50% berberine by weight, or unequal doses of each compound, such as 90% diltiazem and 10% berberine, 80% diltiazem and 20% berberine, 70% diltiazem and 30% berberine, 60% diltiazem and 40% berberine, 40% diltiazem and 60% berberine, 30% diltiazem and 70% berberine, 20% diltiazem and 80% berberine, or even 10% diltiazem and 90% berberine, the percentages being expressed by weight of the compound with respect to total weight of the combination.

### Diltiazem

Diltiazem is a molecule that is a member of the family of benzothiazepines, referenced under the CAS number 42399-41-7.

Within the meaning of the present invention, “diltiazem” designates the molecule in the form of one of its enantiomers L-cis or D-cis, or a racemic mixture of the two, or even a diltiazem salt such as diltiazem hydrochloride, for which the expanded chemical formula is represented below by formula (I):

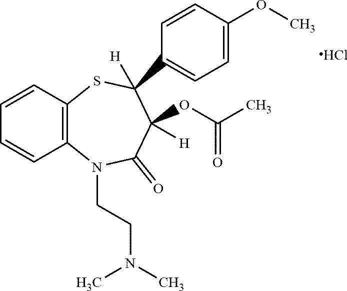 [SMILES: COc1ccc([C@@H]2Sc3ccccc3N(CCN(C)C)C(=O)[C@@H]2OC(C)=O)cc1 ; Cl]

Diltiazem has been known for more than 30 years and is approved, in Europe and in the United States, by the drug regulatory authorities. It can be administered in the form of diltiazem hydrochloride. Cardizem®, Cartia®, Taztia® and Dilacor® are its most common commercial names.

Many formulations are available, in particular prolonged release formulations. Diltiazem is available in various galenic forms, such as in the form of a cream for topical application, in the form of tablets or capsules for oral administration, in the form of powder for the preparation of an injectable solution or in the form of pharmaceutical preparations for inhalation (WO 02/094238, US 4,605,552).

The conventional posology for humans is 180 to 360 mg/day, administered in a capsule or tablets, for the therapeutic use thereof as a calcium channel blocker.

The first identified physiological property of this compound is the blocking of the calcium channels, and therefore the blocking of the intracellular calcium flows. Diltiazem slows, in particular, the transmembrane entry of calcium at the myocardial muscle fibre and the smooth muscle fibre of the blood vessels. This makes it possible to reduce the intracellular calcium concentration reaching the contractile proteins.

In humans, diltiazem administration is indicated for its vasodilatory action, for the purpose of reducing cardiac work. It is thus used to address cardiac and circulatory disorders, such as angina pectoris, arterial hypertension, myocardial ischaemia and tachycardia.

Diltiazem also acts by reversing the effects of angiotensin II, from the renal and peripheral point of view. In topical application, diltiazem can be indicated in the case of chronic anal fissures.

Patent EP 1 117 408 describes the use of diltiazem as a calcium channel blocking compound, for treating pathologies related to the degeneration of the photoreceptors of the retina.

With regard to the use of diltiazem for the treatment of viral infections, as previously stated, this is already described in a plurality of patent applications. Moreover, a chemical trial is currently in progress (FLUNEXT PHRC #15-0442 ClinicalTrials.gov Identifier: NCT03212716), with the aim of obtaining marketing authorisation for this novel therapeutic antiviral indication.

### Berberine

Berberine is a natural alkaloid that is found in a large number of plants, in particular in the species Berberi. Its CAS number is 633-66-9.

Within the meaning of the present invention, “berberine” designates the molecule in all its forms. Its expanded chemical structure is shown schematically below:

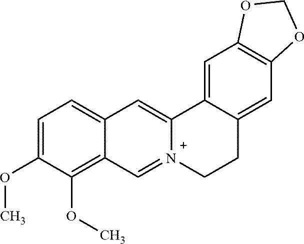 [SMILES: COc1ccc2cc3[n+](cc2c1OC)CCc1cc2c(cc1-3)OCO2]

This molecule is widely used in the Asiatic pharmacopoeia, for its antifungal, antibacterial and anti-inflammatory properties.

In the cells, berberine is located, in particular, in the mitochondria where it inhibits respiratory complex I, thus reducing the production of ATP and the subsequent activation of AMPK (adenosine monophosphate activated protein kinase). This ubiquitous enzyme plays a role in cellular energy homeostasis. The main effect of AMPK activation is (i) to stimulate the oxidation of hepatic fatty acids and ketogenesis, (ii) to inhibit the synthesis of cholesterol, lipogenesis and the synthesis of triglycerides, (iii) to stimulate oxidation of fatty acids in the skeletal muscles and the absorption of glucose by the muscles and (iv) to modulate the secretion of insulin by the beta cells of the pancreas.

The bioavailability of berberine is low, but this does not present difficulties because the systematic action of berberine passes for a large part via either the modification of the intestinal microbiota and its metabolites, or after metabolisation of berberine by this same microbiota.

The half-life of berberine is also low, of order 4 hours. This implies that the daily doses should ideally be divided into 3 intakes. For an adult, the daily dose will generally be from 500 mg to 1500 mg.

### Combination With Another Active Ingredient

It is intended that the composition for use thereof according to the invention comprises at least one compound chosen from diltiazem and berberine, and that it can also comprise other active compounds, in addition to the suitable pharmaceutical carrier.

More specifically, diltiazem and berberine or the mixture thereof, can be used in therapy, alone or in combination with at least one other active ingredient.

This may involve compounds for improving the antiviral activity of diltiazem and/or berberine, or conversely diltiazem and berberine can act as potentiators of these other active compounds.

Thus, the present invention relates to diltiazem or berberine or their combination, for the use thereof in the potentiating of the antiviral effects of other therapeutic compounds used for treating and/or preventing viral infection by the SARS-CoV-2, in particular those cited in the present application.

These additional active compounds may be chosen from the pharmaceutical classes of agents cited in application WO 2015/157223, namely from antibacterial agents, anti-parasite agents, neurotransmission inhibitors, oestrogen receptor inhibitors, inhibitors of the synthesis and replication of DNA, protein maturation inhibitors, kinase pathway inhibitors, cytoskeletal inhibitors, lipid metabolism inhibitors, anti-inflammatory agents, ion channel blockers, apoptosis inhibitors and cathepsin inhibitors.

Hence, according to a particular embodiment of the invention, the pharmaceutical composition for the use thereof as described above comprises at least one other active ingredient, in particular an antiviral agent.

Within the meaning of the present invention, the terms “antiviral agent” or “antiviral compound” shall mean active ingredients which act on the viral load (also referred to as the infectious titre), by inhibiting, either directly or indirectly, the replication and/or dissemination of a virus and in particular, in the present case, of coronavirus SARS-CoV-2, within an infected organism.

According to a particular aspect of the invention, the pharmaceutical composition for the use thereof in the prevention and/or treatment of an infection by coronavirus SARS-CoV-2, comprises, in addition to diltiazem and/or berberine, at least one other antiviral agent.

It is intended that this other antiviral agent will be used in the doses necessary to exhibit an antiviral action, this dose being designated as being “effective”, this dosage being easily determinable by a person skilled in the art.

This combination comprises either the same quantity by weight of each antiviral compound, i.e. a combination of 50% diltiazem and/or berberine and 50% of another antiviral agent by weight, or unequal doses of each compound, such as 90% diltiazem and/or berberine to 10% of the other antiviral agent, 80%-20%, 70%-30%, 60%-40%, 40%-60%, 30%-70%, 20%-80%, or even 10% diltiazem and/or berberine and 90% of another antiviral agent, the percentages being expressed by weight of the compound with respect to the total weight of the combination.

“Antiviral activity” or “antiviral action” shall mean either:
a direct action on the virus, in particular the action of inhibiting the replication cycle of the virus or its capacity to infect and reproduce in the host cells, or
an indirect action on the target cells of said virus, by modulating the expression of certain genes of the target cells. “Target cells” shall mean cells infected by coronavirus and/or likely to be infected next, due to their immediate proximity with the infected cells.

Antiviral agents are classified in various categories according to their mode of action. These include, in particular:
nucleotide or ribonucleoside analogues that interfere with or stop the synthesis of DNA or RNA;
inhibitors of enzymes involved in DNA or RNA synthesis (helicase, replicase);
viral protease inhibitor(s);
compounds that inhibit the maturation steps of the virus during its replication cycle;
compounds that interfere with the binding to the cell membrane, or to the entry of viruses into the host cells (fusion or entry inhibitors) such as transmembrane serine protease inhibitors, in particular those of type 2;
agents that prevent the virus from expressing itself within the host cell after its entry, by blocking its disassembly within the cell;
agents that restrict the propagation of the virus to other cells.

Among these antiviral agents which are well known to a person skilled in the art, those used in particular to combat RNA viruses are: nucleoside analogues, viral protease inhibitor(s), helicase inhibitors and inhibitors of the entry of the virus into the target cells, such as transmembrane serine protease inhibitors.

Within the meaning of the invention, a “nucleoside analogue” means a compound used for preventing viral replication in the infected cells, such as aciclovir. These compounds have structures sufficiently similar to the nucleosides to be incorporated in the viral DNA strands during replication, but they act as chain terminator agents and stop the action of viral DNA polymerase.

Such a compound will be chosen from a nucleoside analogue of guanosine (for example ribavirin), adenosine (for example remdesivir or galidesivir), cytidine (molnupiravir ) or thymidine, or the deoxy- versions thereof.

Within the meaning of the invention, a “viral protease inhibitor” means an antiviral compound acting by inhibiting the action of at least one viral protease, a protein which enables the cleaving and assembly of viral proteins, an indispensable method for obtaining new infectious virions. The virions obtained are then incapable of infecting new cells. This therapeutic strategy is used, in particular, for treating viral infections by HIV (human immunodeficiency virus).

Within the meaning of the invention, a “transmembrane serine protease inhibitor” means an antiviral compound acting by inhibiting the entry of the virus into the cell, in particular by its action on transmembrane serine protease 2, designated by the abbreviation TMPRSS2.

According to a particular embodiment of the invention, the composition for the use thereof as described above comprises at least one other active ingredient chosen from:
a nucleoside analogue;
a viral protease inhibitor;
a transmembrane serine protease inhibitor;
chloroquine, and
any mixture of the above compounds.

According to a first embodiment of the invention, the combination comprises diltiazem and at least one nucleoside analogue.

According to a second embodiment of the invention, the combination comprises diltiazem and at least one viral protease inhibitor.

According to a third embodiment of the invention, the combination comprises diltiazem and at least one transmembrane serine protein inhibitor, in particular of type 2.

According to a fourth embodiment of the invention, the combination comprises diltiazem and chloroquine.

According to a fifth embodiment of the invention, the combination comprises diltiazem and a mixture of these two compounds chosen from:
a nucleoside analogue;
a viral protease inhibitor;
a transmembrane serine protease inhibitor; and
chloroquine.

According to a sixth embodiment of the invention, the combination comprises berberine and at least one nucleoside analogue.

According to a seventh embodiment of the invention, the combination comprises berberine and at least one viral protease inhibitor.

According to an eighth embodiment of the invention, the combination comprises berberine and at least one transmembrane serine protein inhibitor, in particular of type 2.

According to a ninth embodiment of the invention, the combination comprises berberine and chloroquine.

According to a tenth embodiment of the invention, the combination comprises berberine and a mixture of at least two compounds chosen from:
a nucleoside analogue;
a viral protease inhibitor;
a transmembrane serine protease inhibitor; and
chloroquine.

The at least one nucleoside analogue may, in particular, be chosen from the group consisting of: remdesivir, galidesivir, molnupiravir and the combinations thereof.

The at least one viral protease inhibitor may be, in particular, lopinavir, and preferably lopinavir combined with rinotavir.

The at least one transmembrane serine protein inhibitor, in particular of type 2, may be, in particular, camostat mesilate.

According to other embodiments, the pharmaceutical composition for the use thereof according to the invention comprises or consists in:
diltiazem and remdesivir;
diltiazem and galidesivir;
diltiazem and molnupiravir;
diltiazem and lopinavir, preferably combined with rinotavir;
diltiazem and camostat mesilate;
diltiazem and chloroquine, or even
diltiazem and all the possible combinations of remdesivir, galidesivir, molnupiravir, lopinavir, camostat mesilate and chloroquine.

According to other embodiments, the pharmaceutical composition for the use thereof according to the invention comprises or consists in:
berberine and remdesivir;
berberine and galidesivir;
berberine and molnupiravir;
berberine and lopinavir, preferably combined with rinotavir;
berberine and camostat mesilate;
berberine and chloroquine, or even
berberine and all the possible combinations of remdesivir, galidesivir, molnupiravir, lopinavir, camostat mesilate and chloroquine.

According to other embodiments, the pharmaceutical composition for the use thereof according to the invention comprises or consists in:
a combination of diltiazem and berberine, and remdesivir;
a combination of diltiazem and berberine, and galidesivir;
a combination of diltiazem and molnupiravir;
a combination of diltiazem and berberine, and lopinavir, preferably combined with rinotavir;
a combination of diltiazem and berberine, and camostat mesilate;
a combination of diltiazem and berberine, and chloroquine, or even
a combination of diltiazem and berberine, and all the possible combinations of remdesivir, galidesivir, molnupiravir, lopinavir, camostat mesilate and chloroquine.

These combinations have synergistic antiviral effects, as shown in the examples.

For example, the results presented in table 2 highlight the fact that, at 48 hours post-infection, the effects of remdesivir are greater (+68%) in the presence of diltiazem, as well as in thepresence of berberine (+33%).

The results presented in table 3 highlight that, on the nasal epithelium models, 48 hours post-infection, the effects of remdesivir are potentiated by the presence of diltiazem (+1.3 log reduction in viral production) or of berberine (+0.89 log reduction in viral production).

According to another embodiment of the invention, the composition for the therapeutic use thereof, as described above, additionally comprises at least one antibiotic.

Such an antibiotic will, in particular, be useful for preventing the bacterial superinfection with the ongoing viral infection.

The antibiotic is chosen from the antibiotics well known to a person skilled in the art, in particular those used during viral infections in order to avoid bacterial superinfection, and in particular those of the macrolide family.

The pharmaceutical compositions according to the present invention are suitable for nasal, oral, sublingual, inhalation, subcutaneous, intramuscular, intravenous, transdermal, ocular or rectal administration.

According to a preferred embodiment, the composition for the use thereof as described above is characterised in that it is in a galenic form suitable for a nasal administration, in particular intranasal administration, in particular by inhalation.

The intranasal route is an administration route characterised in that the pharmaceutical composition is introduced directly into the nasal cavity of the patient, by various methods, for example: drops, spray, or inhaler. The use of a specific device, such as an intranasal mucosal spray device, is recommended.

The intranasal route offers the possibility of administering a drug rapidly, painlessly and non-invasively, with an efficacy that is often comparable to that of the intravenous route. It is particularly suitable in paediatrics or for elderly persons, or in medical emergency situations.

According to an embodiment, the pharmaceutical composition for the use thereof as described above is administered by the intranasal route.

According to another preferred embodiment, the pharmaceutical composition for the use thereof as described above is administered by inhalation.

Inhalation designates absorption by the respiratory tract. This is, in particular, a method for absorbing compounds for therapeutic purposes and certain substances in the form of a gas, micro-droplets or powder in suspension.

The administration of pharmaceutical or veterinary compositions by inhalation, i.e. by the nasal and/or oral route, is well known to a person skilled in the art.

There are two types of administration by inhalation:
administration by insufflation when the compositions are in the form of powders, and
administration by atomisation when the compositions are in the form of aerosols (suspensions) or in the form of pressurised solutions, for example aqueous solutions. The use of an atomiser or spray is then recommended for administering the pharmaceutical or veterinary composition.

The galenic form considered here is therefore chosen from: a powder, an aqueous suspension of droplets or a pressurised solution.

The present invention also relates to a combination product comprising at least one compound chosen from diltiazem and berberine, and at least one other active ingredient chosen from:
a nucleoside analogue;
a viral protease inhibitor;
a transmembrane serine protease inhibitor;
chloroquine, and
any mixture of the above compounds,
for the simultaneous, separated or sequential use thereof in the prevention and/or treatment of a viral infection by the SARS-CoV-2 virus (COVID-19).

This combination product will, in particular, consist of:
diltiazem and remdesivir;
diltiazem and galidesivir;
diltiazem and molnupiravir;
diltiazem and lopinavir;
diltiazem and camostat mesilate;
diltiazem and chloroquine, or even
diltiazem and all the possible combinations of remdesivir, galidesivir, molnupiravir, lopinavir, camostat mesilate and chloroquine.

According to other embodiments, this combination product will, in particular, consist of:
berberine and remdesivir;
berberine and galidesivir;
berberine and molnupiravir;
berberine and lopinavir;
berberine and camostat mesilate;
berberine and chloroquine, or even
berberine and all the possible combinations of remdesivir, galidesivir, molnupiravir, lopinavir, and chloroquine.

According to other embodiments, this combination product will, in particular, consist of:
a combination of diltiazem and berberine, and remdesivir;
a combination of diltiazem and berberine, and galidesivir;
a combination of diltiazem and berberine, and molnupiravir;
a combination of diltiazem and berberine, and lopinavir;
a combination of diltiazem and berberine, and camostat mesilate;
a combination of diltiazem and berberine, and chloroquine, or even
a combination of diltiazem and berberine, and all the possible combinations of remdesivir, galidesivir, molnupiravir, lopinavir, camostat mesilate and chloroquine.

According to an embodiment according to the invention, one of these combination products as described above is simultaneously, separately or sequentially used for the prevention of a viral infection by the SARS-CoV-2 virus (COVID-19).

According to another embodiment according to the invention, one of these combination products as described above is simultaneously, separately or sequentially used for the treatment of a viral infection by the SARS-CoV-2 virus (COVID-19).

This combination product may comprise other active compounds, and in particular at least one antibiotic.

The present invention also relates to a method for treating a patient infected by a SARS-CoV-2 virus (suffering from so-called COVID-19 disease) comprising the administration to said patient of a pharmaceutical composition comprising, in a suitable pharmaceutical carrier, at least one compound chosen from diltiazem and berberine.

The present invention also relates to a method for preventing the appearance of a viral infection by a SARS-CoV-2 virus (so-called COVID-19 disease), in an individual able to be infected by said virus, comprising the administration to said individual of a pharmaceutical composition comprising diltiazem in a suitable pharmaceutical carrier.

The present invention also relates to a method for treating a patient infected by a SARS-CoV-2 virus (suffering from so-called COVID-19 disease) comprising the administration to said patient of a pharmaceutical composition comprising diltiazem in a suitable pharmaceutical carrier.

In particular, this method can also comprise the administration, to said patient, of another active compound, in particular chosen from the following compounds:
a nucleoside analogue;
a viral protease inhibitor;
a transmembrane serine protease inhibitor;
chloroquine, and
any mixture of the above compounds.

The present invention also relates to a pharmaceutical composition comprising, in a suitable pharmaceutical carrier, a combination of diltiazem and/or berberine, with remdesivir.

The present invention also relates to a pharmaceutical composition comprising, in a suitable pharmaceutical carrier, a combination of diltiazem with remdesivir.

The present invention also relates to a pharmaceutical composition comprising, in a suitable pharmaceutical carrier, a combination of diltiazem with molnupiravir.

More precisely, said pharmaceutical composition comprises, in a suitable pharmaceutical carrier, a combination of diltiazem, berberine, and remdesivir.

The present invention also relates to pharmaceutical compositions comprising the following combinations, in a suitable pharmaceutical carrier:
a combination of diltiazem and berberine, and galidesivir;
a combination of diltiazem and berberine, and lopinavir;
a combination of diltiazem and berberine, and molnupiravir;
a combination of diltiazem and berberine, and camostat mesilate;
a combination of diltiazem and berberine, and chloroquine, even
a combination of diltiazem and berberine, and all the possible combinations of remdesivir, galidesivir, molnupiravir, lopinavir, camostat mesilate and chloroquine.

These combinations may be formulated according to all the possible ratios of each compound. In particular, they comprise either the same quantity by weight of each antiviral compound (33% of each), or unequal doses of each compound, the percentages being expressed by weight of the compound with respect to the total weight of the combination.

All the therapeutic uses of these combinations are also objects of the invention.

### EXAMPLES

Examples 1 to 3 show, on the one hand, the effectiveness of a treatment with diltiazem or with berberine in monotherapy and, on the other hand, the advantage of using these molecules in combination with remdesivir for the treatment of infections with SARS-CoV-2.

Other data, not presented here, have been obtained using apigenin, another compound having an antiviral action on certain viral strains, in particular against MERS-CoV (WO 2018/073549). However, this molecule does not have a significant antiviral action against coronavirus SARS-CoV-2.

The viral load was quantified by RT-qPCR and/or by TCID50/ml in samples of the Vero E6 cell infection supernatant, but also in apical washes and reconstituted human epithelium cell lysates (HAE MucilAir, Epithelix), treated with diltiazem, berberine or remdesivir, in monotherapy or in combinations of two of these molecules (diltiazem+remdesivir, or berberine+remdesivir).

The viral production relative to each of the concentrations of molecules has been determined and is shown with respect to the viral production from infected cells or HAE under the same conditions but untreated. The median inhibitory concentrations (IC50) were determined for each treatment condition.

### Example 1. Materials and Methods

### Used Virus

The used virus was isolated from a sample from a patient infected by SARS-CoV-2.

The strain of SARS-CoV-2 used in this study was isolated from a 47 year old patient, recruited in a French clinical cohort evaluating patients with COVID-19 (NCT04262921). This study was carried out in compliance with the Helsinki declaration and was approved by the local ethics committee. The viral strain was sequenced with Illumina MiSeq and deposited in the GISAID EpiCoVTM database under reference BetaCoV/France/IDF0571/2020 (identification number EPI_ISL_411218). For reference, see (Pizzorno et al., 2020).

The isolation of the virus was carried out by inoculation with Vero E6 cells (ATCC CRL-1586) and followed by the appearance of cytopathic effects. After appearance of the first effects induced by the virus, the infection supernatant was harvested and the viral RNA was extracted using the QIAmp Viral RNA (QIAGEN) kit. The extracted RNA was then subjected to sequencing by Illumina MiSeq (Zymo-Seq RiboFree), with a coverage of 500×, and the sequences were assembled using hisat2 alignment programs and consensus.

The sequence was then filed on the GISAID EpiCoV platform (Accession ID EPI_ISL_411218) under the name BetaCoV/France/IDF0571/2020.

This viral strain is phylogenetically very close to SARS-CoV-2 strains which circulated at the start of the epidemic in the Wuhan region of China in the months of January/February 2020. This strain is therefore representative of the SARS-CoV-2 strains at the origin of the infectious diseases referred to as “COVID-19” currently observed in the world.

### Vero E6 Cell Infection Protocol

The Vero E6 (ATCC CRL-1586) cells were cultivated in medium DMEM 4.5 g/l of glucose, supplemented by L-glutamine and penicillin/streptomycin and 10% inactivated foetal calf serum, at 37° C., 5% CO2.

In order to produce the infections, the cells were rinsed twice by the medium without serum and were covered by a minimum volume containing a dilution of virus, this dilution being determined from the infectious titre (cf. section on determination of the infectious titre), in order to obtain an adequate multiplicity of infection (MOI). After incubation for one hour in the presence of a minimum volume, the medium was replaced by the medium DMEM 4.5 g/l of glucose, supplemented by L-glutamine and penicillin/streptomycin and 2% inactivated foetal calf serum, and the cells were incubated again at 37° C., 5% CO2.

### Epithelium Infection Protocol

In order to produce the infections, we have also used a reconstituted human epithelium model (HAE MucilAir, Epithelix), obtained from primary human cells obtained by nasal biopsies, cultivated at the air-liquid interface with a specific culture medium in Costar Transwell inserts (Corning, NY, USA). For the infection experiments, the apical poles were gently washed twice using the medium OptiMEM (Gibco, ThermoFisher Scientific), then infected with 150 µl of virus dilution in medium OptiMEM, with a multiplicity of infection (MOI) of 0.1. After incubation for one hour at 37° C., 5% CO2, the viral suspension was withdrawn.

### Determination of the Infectious Titre in Vero E6 Cell

The determination of the infectious titre was performed using a limited dilution technique, on these Vero E6 cells on a 96-well plate. A volume of 50 µl of series dilutions was deposited in the wells in quadruplicates. The cells were then incubated at 37° C., 5% CO2 and the presence of cytopathic effects is then monitored after 3 days of infection. The infectious dose in tissue culture 50% (DICT50/ ml), i.e. the viral titre required to form an infection in 50% of inoculated cells, was calculated using the technique of Reed and Muench.

### Quantification of the Viral Genome by Quantitative PCR

The probes and primers used have been described by the School of Public Health/University of Hong Kong (table 1).

A quantitative “one-step” PCR was performed using the StepOnePlus Real Time PCR System kit (Applied Biosystems), with the reagent EXPRESS One-Step Superscript qRT-PCR (Invitrogen), in a reaction volume of 20 µl containing 10 µl of supermix Express qPCR (2×), 1 µl of each primer (10 µM), 3.1 µl of water, 0.4 µl Rox dye (25 µM) and 2 µl of viral RNA.

The following program was used: 15 minutes at 50° C., followed by 40 cycles (15 s 95° C.; 1 min 60° C.).

TABLE 1

| Target ORF1b~nsp14 | | SEQ ID NO. |
|---|---|---|
| Oligonucleotide sense (HKU-ORF1b-nsp14F) | 5′-TGGGGYTTTACRGGTAACCT–3′ | 1 |
| Oligonucleotide antisense (HKU- ORF1b-nsp14R) | 5′-AACRCGCTTAACAAAGCACTC-3′ | 2 |
| Probe (HKU-ORF1b-nsp141P) | 5′-FAM-TAGTTGTGATGCWATCATGACTAGTAMRA-3′ | 3 |

FAM and TAMRA designate fluorescent markers.

### Example 2. Comparison of the IC50 of Diltiazem, Berberine and Remdesivir, and Of Diltiazem+Remdesivir and Berberine+Remdesivir Combinations on the SARS-CoV-2 Virus in the Vero E6 Cell Model

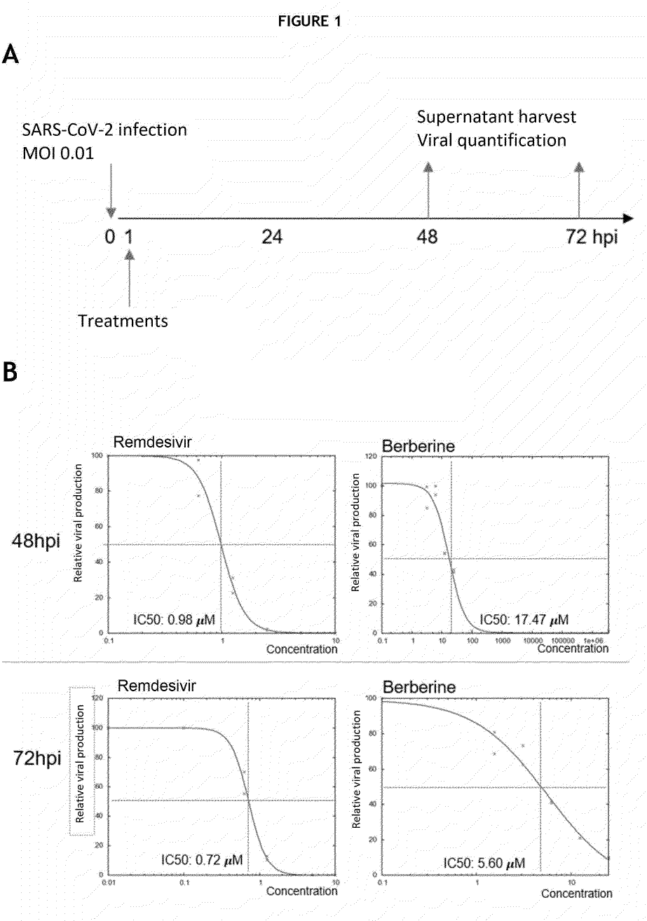

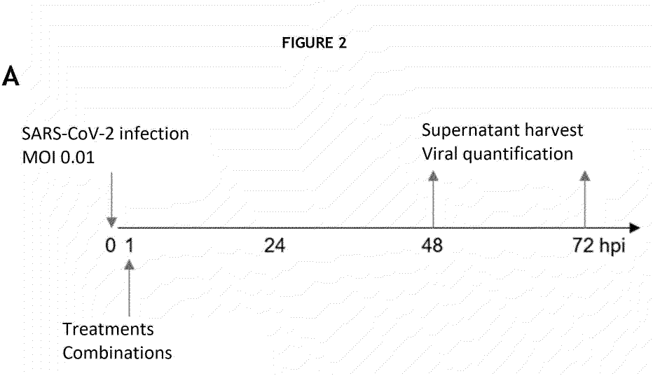

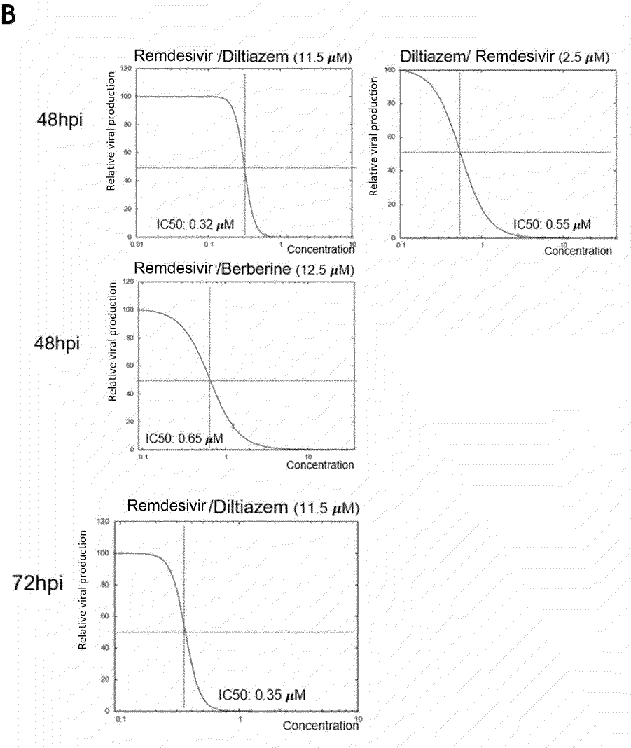

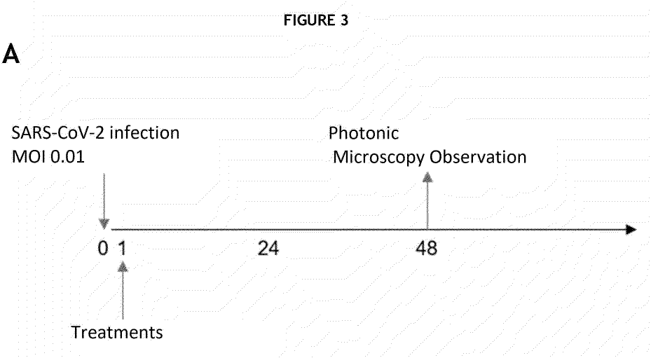

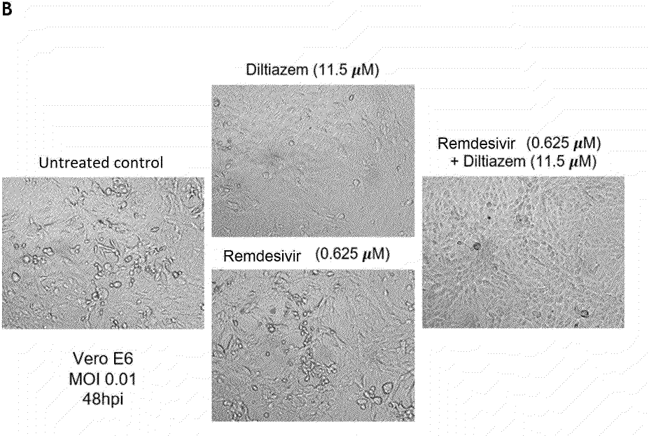

Vero E6 cells infected by SARS-CoV-2 (MOI 0.1) were treated at one hour post-infection by increasing concentrations of:
diltiazem (2.8 to 45 µM),
berberine (1.6 to 25 µM), and
remdesivir (0.6 to 10 µM),
alone (FIG. 1) and in combination (FIG. 2).

For each of the combined treatments tested, a dose of one of the molecules was fixed and combined with increasing concentrations of the other molecule (same concentration ranges as those used in monotherapy, respectively) and vice versa. The viral infectious titres measured in the culture supernatants of the infected cells (sampled at 48 and 72 hours post-infection) reflect the level of viral replication measured under the various treatment conditions.

Table 2 below summarises the IC50 data for different monotherapy treatments and/or obtained in combination in Vero E6 cells at various times of infection by SARS-CoV-2.

TABLE 2

| Experimental conditions | | IC50 (µM) |
|---|---|---|
| MOI 0.01 - 48 hpi | Remdesivir | 0.98 +/- 0.07 |
| | Diltiazem | > 45 |
| | Berberine | 17.47 +/- 3.43 |
| | Remdesivir/diltiazem (11.5 µM) | 0.32 +/- 0.06 |
| | Diltiazem/remdesivir (2.5 µM) | 0.55 +/- 0.25 |
| | Remdesivir/berberine (12.5 µM) | 0.65 +/- 0.04 |
| MOI 0.01 - 72 hpi | Remdesivir | 0.72 +/- 0.03 |
| | Diltiazem | > 45 |
| | Berberine | 5.60 +/- 1.70 |
| | Remdesivir/diltiazem (11.5 µM) | 0.35 +/- 1.20 |

This table illustrates the gain from combinations of treatments in terms of reducing the IC50 of certain molecules in comparison with monotherapy treatments with these molecules.

Hence, remdesivir combined with a fixed concentration of diltiazem (11.5 µM) can obtain a reduction of approximately 68% in its IC50 (0.32 versus 0.98 µM) at 48 hpi, and 52% in its IC50 (0.35 versus 0.72 µM) at 72 hpi.

In parallel, diltiazem combined with a fixed concentration of remdesivir makes it possible to obtain an IC50 of 0.55 µM, whereas its IC50 is greater than 45 µM at 48 hpi.

Furthermore, remdesivir combined with a fixed concentration of berberine (12.5 µM) can obtain a reduction of approximately 33% of its IC50 (0.65 versus 0.98 µM) at 48 hpi.

### Example 3. Comparison of the Antiviral Activities of Diltiazem, Berberine and Remdesivir, and of Diltiazem+Remdesivir, Berberine+Remdesivir Combinations on the SARS-CoV-2 Virus by Reconstituted Human Respiratory Epithelium Infection Model

Reconstituted human respiratory epithelia of nasal origin and cultivated at the air-liquid interface (MucilAir® HAE, Epithelix) were infected with SARS-CoV-2 (MOI 0.1). The basal medium was treated once per day by the following molecules, in simple or combination treatment:
diltiazem (45 or 90 µM),
   remdesivir (20 or 40 µM) and
   berberine (4 µM)
for 48 or 72 hours post-infection.

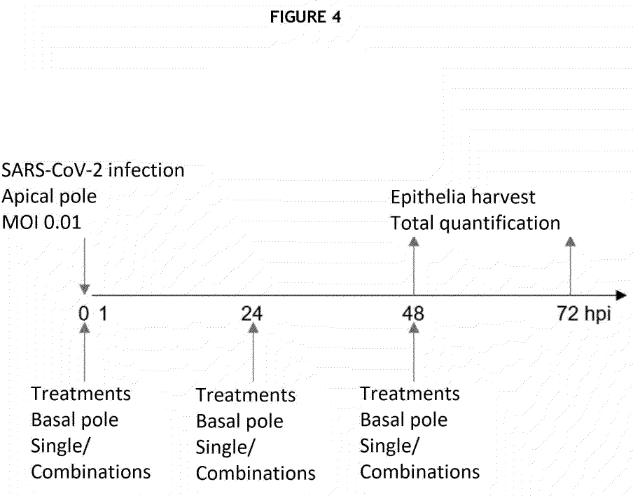

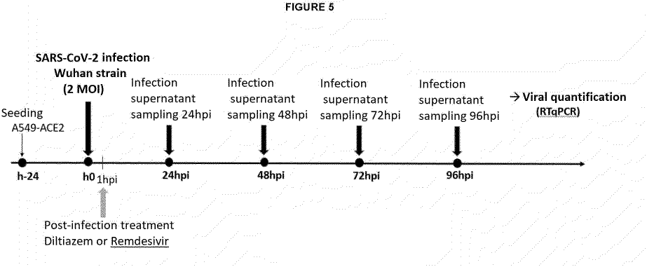

FIG. 4 shows the timing diagram of these experiments.

At various times, the epithelia were harvested and lysed. The total RNA was extracted and the viral genomes were quantified by RT-PCR by normalising the data using the quantification of the product of a cellular gene (GAPDH).

These data make it possible to evaluate the active viral effect of monotherapy treatments and combination treatments by relative measurement of the viral replication, expressed in percentage of the viral replication in the control (not treated) or in -log10 of the relative viral production.

TABLE 3

| Experimental conditions | | Reduction Relative viral production (%) | Reduction Relative viral production (-Iog10) |
|---|---|---|---|
| Epithelium Nasal MOI 0.1 48 hpi | remdesivir (20 µM) | 99.99999555 | 7.3511976275 |
| | diltiazem (90 µM) | 55.12994353 | 0.348043384 |
| | berberine (4 µM) | 86.3693311 | 0.865482832 |
| | remdesivir (20 µM) diltiazem (90 µM) | 99.99999978 | 8.664459134 |
| | remdesivir (20 µM) berberine (4 µM) | 99.99999945 | 8.261020351 |
| Nasal Epithelium MOI 0.1 72 hpi | remdesivir (20 µM) | 99.62026862 | 2.420523516 |
| | remdesivir (40 µM) | 99.49931193 | 2.300432754 |
| | diltiazem (45 µM) | 31.35002452 | 0.163359614 |
| | diltiazem (90 µM) | 29.36760102 | 0.150996043 |
| | remdesivir (20 µM) diltiazem (90 µM) | 99.43372357 | 2.246971516 |
| | remdesivir (40 µM) diltiazem (45 µM) | 99.45500851 | 2.263610278 |

Table 3 shows the effects of monotherapy treatments with diltiazem, with berberine and with remdesivir and combined treatments with diltiazem+remdesivir and with berberine+remdesivir on the replication of the SARS-CoV-2 virus in a human respiratory epithelium model (MucilAir® HAE, Epithelix) of nasal origin.

Table 3 summarises the data of antiviral activity of diltiazem, berberine, remdesivir, and of the diltiazem/remdesivir and berberine/remdesivir combinations on the SARS-CoV-2 virus in a reconstituted human respiratory epithelial infection model, of nasal or bronchial origin.

Remdesivir, in a single treatment, has a significant efficacy at 48 hpi in epithelium of nasal origin (more than 7.75 log10 reduction in viral replication).

The combination of remdesivir with diltiazem or with berberine (under the same concentration conditions as for monotherapy) significantly increases the antiviral effect (8.6 and 8.29 log10 reductions in viral replication for the combination with diltiazem and berberine, respectively).

The monotherapy treatments with diltiazem or berberine also have significant antiviral efficacies at 48 hpi (0.35 and 0.86 log10 of reductions in viral replication with diltiazem and berberine, respectively).

The efficacy of remdesivir at 72 hpi, under our experimental conditions, although remaining very high, is comparatively smaller than at 48 hpi in epithelium of nasal origin (2.42 and 2.05 log10 reduction at 20 µM and 2.30 and 2.24 log 10 reduction at 40 µM).

At 72 hpi, the remdesivir/diltiazem combinations have an efficacy in epithelium of nasal origin, but without significant difference from a simple treatment by remdesivir. This could be explained by an antiviral efficacy of diltiazem that is limited under these experimental conditions (0.16/0.15 Iog10 reduction in nasal epithelium).

### Example 4. Comparison of the Antiviral Activities of Diltiazem and Remdesivir, on the SARS-CoV-2 Virus in an A549-ACE2 Cellular Model

The cells of the cell line A549 are human alveolar basal epithelial cells derived from adenocarcinoma. This cell line is used as a model for the study of lung cancer, but also as target cells for infectious viruses targeting the respiratory tract. These cells were subsequently modified to express the ACE2 receptor, via which the SARS-CoV-2 virus penetrates into the hosts cells. This cell line A549-ACE2 was obtained from Creative Biogene (USA).

Contrary to the Vero cells, these cells A549-ACE2 have a complete and operational signalling pathway for interferons. They are therefore more suitable for studying the effects of diltiazem which acts on these signalling pathways.

The experimental protocol is the following:
Seeding of the A549-ACE2 cells,
24 hours after infection of the A549-ACE2 cells with a Wuhan-like strain of SARS-CoV-2 (MOI 10⁻¹ and 10⁻²),
1 hour post infection (pi), the cells are treated with 45 uM diltiazem.
The supernatants are sampled at 24 hours, 48 hours, 72 hours and 96 hours after the infection for a viral quantification by RT-PCR.

The incubation media used are the following:
Seeding medium: DMEM 1 g/L glucose (Glc), 200 mM L-Glutamine (L-Glu), 104 units penicillin/streptomycin (P/S), 10% foetal calf serum (FCS)
Infection medium: DMEM 1 g/L Glc, 200 mM L-Glu, 104 U P/S, 0% FCS
Treatment medium: DMEM 1 g/L Glc, 200 mM L-Glu, 104 U P/S, 2% FCS

FIG. 5 shows the experimental protocol: a single treatment is performed at 1 hpi, then the supernatant is sampled at 24, 48, 72 and 96 hpi.

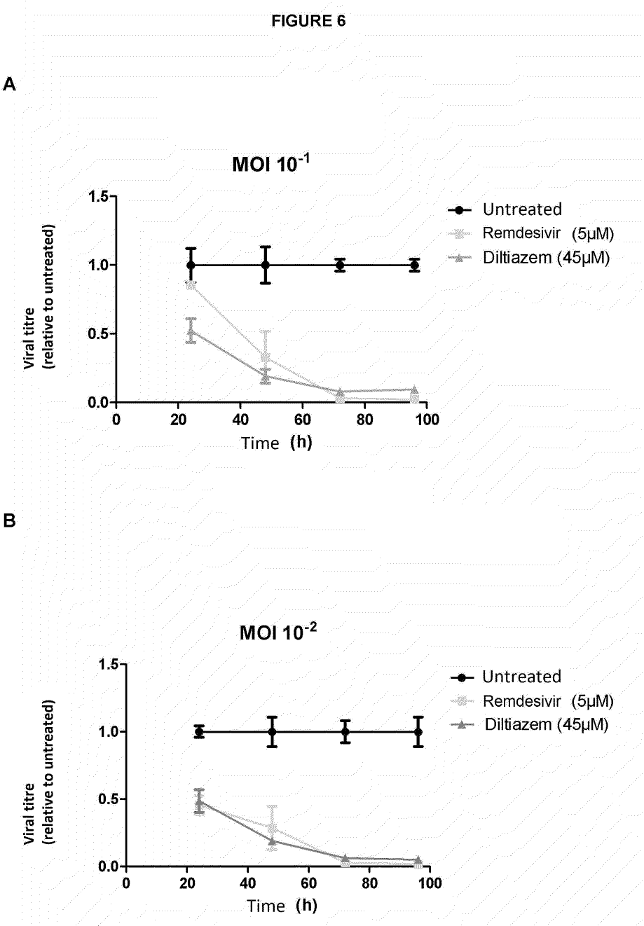

FIG. 6 shows the results obtained after treatment of infected cells at two different infection multiplicity rates (A: MOI = 10⁻¹; B: MOI=10⁻²) with diltiazem (45 µM) or remdesivir (5 µM).

The two compounds remdesivir and diltiazem can obtain, with a single treatment, a same level of inhibition of the viral titre (measured by RT-PCR) over time, and this regardless of the infection multiplicity rate.

### Example 5. Determination of the IC50 on SARS-CoV-2 and the CC50 of Diltiazem, in line A549 ACE2

The experimental protocol is the following:
Seeding of the A549-ACE2 cells,
24 hours after infection of A549-ACE2 cells with a Wuhan-like strain of SARS-CoV-2 (MOI 10⁻¹),
1 hour post infection (pi), the cells are treated with diltiazem at different concentrations.
The supernatants are sampled at 72 hours after the infection for a viral quantification by RT-PCR.

The IC50 is the median inhibitory concentration, i.e. the quantity of diltiazem necessary to obtain 50% inhibition of the viral replication of the tested SARS-CoV-2 strain.

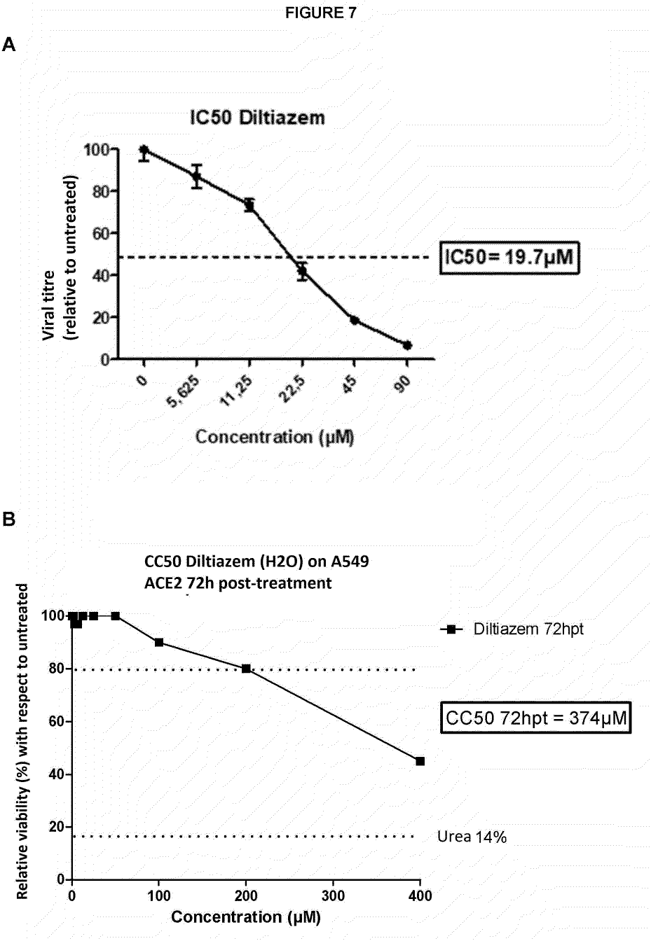

The results obtained are presented in FIG. 7A: the IC50 of diltiazem is 19.7 µM, which signifies a good inhibitory efficacy in vitro.

In parallel, the cytotoxicity of diltiazem on A549-ACE2 cells has been verified under the same experimental conditions. For this purpose, the CC50 (cytotoxic concentration 50%), which corresponds to the necessary dose of diltiazem in order to reduce the viability of the cells by half, was measured by monitoring the viability of the A549-ACE2 cells in the presence of various concentrations of diltiazem. The variability was determined by an MTS carried out 72 hours after the start of the treatment.

The MTS test is a colorimetric method: the method is based on the reduction of the compound MTS tetrazolium by viable cells in order to generate a coloured formazan product, enabling the counting of viable cells (coloured) and dead cells.

The results obtained are shown in FIG. 7B: the viability of the cells is reduced by half with a diltiazem concentration equal to 374 µM, a dose very much higher than the previously measured IC50.

### BIBLIOGRAPHIC REFERENCES IN THE ORDER OF CITATION IN THE DESCRIPTION

### Patents

EP 2 435 064
WO 87/07508
WO 2011/066657
WO 2016/146836
WO 2019/224489
WO 2013/185126
WO 2018/073549
WO 02/094238
US 4,605,552
EP 1 117 408
WO 2015/157223

### Articles

Alexander E. Gorbalenya, Susan C. Baker, Ralph S. Baric, Raoul J. de Groot, Christian Drosten, Anastasia A. Gulyaeva, Bart L. Haagmans, Chris Lauber, Andrey M Leontovich, Benjamin W. Neuman, Dmitry Penzar, Stanley Perlman, Leo L.M. Poon, Dmitry Samborskiy, Igor A. Sidorov, Isabel Sola and John Ziebuhr, “Severe acute respiratory syndrome-related coronavirus - The species and its viruses, a statement of the Coronavirus Study Group”, bioRxiv, 2020

Zhou P, Yang XL, Wang XG, Hu B, Zhang L, Zhang W, Si HR, Zhu Y, Li B, Huang CL, Chen HD, Chen J, Luo Y, Guo H, Jiang RD, Liu MQ, Chen Y, Shen XR, Wang X, Zheng XS, Zhao K, Chen QJ, Deng F, Liu LL, Yan B, Zhan FX, Wang YY, Xiao GF, Shi ZL. A pneumonia outbreak associated with a new coronavirus of probable bat origin. Nature. 2020 February 3. doi: 10.1038/s41586-020-2012-7

Hui DS, I Azhar E, Madani TA, Ntoumi F, Kock R, Dar O, Ippolito G, Mchugh TD, Memish ZA, Drosten C, Zumla A, Petersen E. The continuing 2019-nCoV epidemic threat of novel coronaviruses to global health - The latest 2019 novel coronavirus outbreak in Wuhan, China. Int J Infect Dis. 2020 February; 91:264-266. doi: 10.1016/j.ijid.2020.01.009. Epub 2020 January 14. PubMed PMID: 31953166

Lo MK, Jordan R, Arvey A, Sudhamsu J, Shrivastava-Ranjan P, Hotard AL, Flint M, McMullan LK, Siegel D, Clarke MO, Mackman RL, Hui HC, Perron M, Ray AS, Cihlar T, Nichol ST, Spiropoulou CF. GS-5734 and its parent nucleoside analog inhibit Filo-, Pneumo-, and Paramyxoviruses. Sci Rep. 2017 March 6; 7:43395. doi:10.1038/srep43395. PubMed PMID: 28262699; PubMed Central PMCID: PMC5338263

Painter WP, Holman W, Bush JA, Almazedi F, Malik H, Eraut NCJE, Morin MJ, Szewczyk LJ, Painter GR. Human Safety, Tolerability, and Pharmacokinetics of Molnupiravir, a Novel Broad-Spectrum Oral Antiviral Agent with Activity Against SARS-CoV-2. Antimicrob Agents Chemother. 2021 March 1:AAC.02428-20.

Sheahan TP, Sims AC, Zhou S, Graham RL, Pruijssers AJ, Agostini ML, Leist SR, Schafer A, Dinnon KH 3rd, Stevens LJ, Chappell JD, Lu X, Hughes TM, George AS, Hill CS, Montgomery SA, Brown AJ, Bluemling GR, Natchus MG, Saindane M, Kolykhalov AA, Painter G, Harcourt J, Tamin A, Thornburg NJ, Swanstrom R, Denison MR, Baric RS. An orally bioavailable broad-spectrum antiviral inhibits SARS-CoV-2 in human airway epithelial cell cultures and multiple coronaviruses in mice. Sci Transl Med. 2020 April 29; 12(541):eabb5883.

Chu CM, Cheng VC, Hung IF, Wong MM, Chan KH, Chan KS, Kao RY, Poon LL, Wong CL, Guan Y, Peiris JS, Yuen KY; HKU/UCH SARS Study Group. Role of lopinavir/ritonavir in the treatment of SARS: initial virological and clinical findings. Thorax. 2004 March; 59(3):252-6. PubMed PMID: 14985565; PubMed Central PMCID: PMC1746980

Hoffmann M, Kleine-Weber H, Schroeder S, Krüger N, Herrler T, Erichsen S, Schiergens TS, Herrler G, Wu NH, Nitsche A, Müller MA, Drosten C, Pöhlmann S.SARS-CoV-2 Cell Entry Depends on ACE2 and TMPRSS2 and Is Blocked by a Clinically Proven Protease Inhibitor. Cell. 2020 March 4. pii: S0092-8674(20)30229-4. doi:10.1016/j.cell.2020.02.052. [Epub ahead of print] PubMed PMID: 32142651

Vincent MJ, Bergeron E, Benjannet S, Erickson BR, Rollin PE, Ksiazek TG, Seidah NG, Nichol ST. Chloroquine is a potent inhibitor of SARS coronavirus infection and spread. Virol J. 2005 August 22; 2:69. PubMed PMID: 16115318; PubMed Central PMCID: PMC1232869

Wang, M., Cao, R., Zhang, L. et al. Remdesivir and chloroquine effectively inhibit the recently emerged novel coronavirus (2019-nCoV) in vitro. Cell Res 30, 269-271 (2020)

Gao J, Tian Z, Yang X. Breakthrough: Chloroquine phosphate has shown apparent efficacy in treatment of COVID-19 associated pneumonia in clinical studies. Biosci Trends. 2020 February 19. doi: 10.5582/bst.2020.01047. [Epub ahead of print] PubMed PMID: 32074550

Fujioka Y, Nishide S, Ose T, Suzuki T, Kato I, Fukuhara H, Fujioka M, Horiuchi K, Satoh AO, Nepal P, Kashiwagi S, Wang J, Horiguchi M, Sato Y, Paudel S, Nanbo A, Miyazaki T, Hasegawa H, Maenaka K, Ohba Y. A Sialylated Voltage-Dependent Ca2+ Channel Binds Hemagglutinin and Mediates Influenza A Virus Entry into Mammalian Cells. Cell Host Microbe. 2018 June 13; 23(6):809-818.

Mohan MC, Abhimannue AP, B PK. Identification and Characterization of Berberine in Tinospora cordifolia by Liquid Chromatography Quadrupole Time of Flight Mass Spectrometry (LC MS/MS Q-tof) and Evaluation of its anti Inflammatory Potential. Pharmacognosy Journal. 2017; 9(3):350-355

Song S, Qiu M, Chu Y, Chen D, Wang X, Su A, Wu Z. Berberine down-regulates cellular JNK and NF-κB activation and this may result in an inhibition of HSV replication. Antimicrob Agents Chemother. 2014 June 9

Wu Y, Li JQ, Kim YJ, Wu J, Wang Q, Hao Y. In vivo and in vitro antiviral effects of berberine on influenza virus. Chin J Integr Med. 2011 June; 17(6):444-52

Anna Luganini, Beatrice Mercorelli, Lorenzo Messa and Giorgio Palù, “The isoquinoline alkaloid berberine inhibits human cytomegalovirus replication by interfering with the viral Immediate Early-2 (IE2) protein transactivating activity. ”, Antiviral Research, vol. 164, April 2019, p. 52-60

Ting-Chun Hung, Alagie Jassey, Ching-Hsuan Liu and Chien-Ju Lin, “Berberine inhibits hepatitis C virus entry by targeting the viral E2 glycoprotein”, Phytomedicine, vol. 53, February 2019, p. 62-69

Mariana Batista, Ana Braga, Guilherme Campos and Marcos Souza, “Natural Products Isolated from Oriental Medicinal Herbs Inactivate Zika Virus”, Viruses, vol. 11, no 1, 11 Jan. 2019, p. 49.

Andres Pizzorno, Blandine Padey, Thomas Julien, Sophie Trouillet-Assant, Aurelien Traversier, Elisabeth Errazuriz-Cerda, Julien Fouret, Julia Dubois, Alexandre Gaymard, François-Xavier Lescure, Victoria Dulière, Pauline Brun, Samuel Constant, Julien Poissy, Bruno Lina, Yazdan Yazdanpanah, Olivier Terrier and Manuel Rosa-Calatrava. “Characterization and treatment of SARS-CoV-2 in nasal and bronchial human airway epithelia.” 2020 Cell Reports Medecine Volume 1, Issue 4, 21 Jul. 2020, 100059

1. A method for treating a patient infected by a SARS-CoV-2 virus or for preventing the appearance of a viral infection by a SARS-CoV-2 virus in an individual susceptible to be infected by said virus, comprising the administration to said patient or individual of a pharmaceutical composition comprising diltiazem in a suitable pharmaceutical carrier .

2. (canceled)

3. The method according to claim 1, wherein the pharmaceutical composition further comprises at least one other active ingredient chosen from:
a nucleoside analogue;
a viral protease inhibitor;
a transmembrane serine protease inhibitor;
chloroquine, and
any mixture of the above compounds.

4. The method according to claim 3, wherein the pharmaceutical composition further comprises a nucleoside analogue chosen from remdesivir, galidesivir, molnupiravir and the combinations thereof.

5. The composition for the use thereof method according to claim 4, wherein the pharmaceutical composition further comprises a combination of diltiazem and remdesivir.

6. The method according to claim 4, wherein the pharmaceutical composition further comprises a combination of diltiazem and galidesivir.

7. The method according to claim 4, wherein the pharmaceutical composition further comprises a combination of diltiazem and molnupiravir.

8. The method according to claim 3, wherein the pharmaceutical composition further comprises a viral protease inhibitor, in particular lopinavir, preferably lopinavir combined with rinotavir.

9. The method according to claim 3, wherein the pharmaceutical composition further comprises a transmembrane serine protease inhibitor, in particular camostat mesilate.

10. The method according to claim 1, additionally comprising the administration to said patient or individual of at least one antibiotic.

11. The method according to claim 1, wherein said pharmaceutical composition is in a galenic form suitable for an intranasal administration, in particular by inhalation.

12. The method according to claim 1, comprising the simultaneous, separate or sequential administration of a combination product comprising diltiazem and at least one other active ingredient chosen from:
a nucleoside analogue;
a viral protease inhibitor;
a transmembrane serine protease inhibitor;
chloroquine, and
any mixture of the above compounds,
for to a patient infected by a SARS-CoV-2 virus or to an individual susceptible to be infected by said virus .

13. A pharmaceutical composition comprising, in a suitable pharmaceutical carrier, a combination of diltiazem and remdesivir.

14. A pharmaceutical composition comprising, in a suitable pharmaceutical carrier, a combination of diltiazem and molnupiravir.


SEQUENCE LISTING (3 sequences)

SEQ ID NO: 1 - DNA, 20 bp, Artificial Sequence
  Oligonucleotide
tggggytttacrggtaacct

SEQ ID NO: 2 - DNA, 21 bp, Artificial Sequence
  Oligonucleotide
aacrcgcttaacaaagcactc

SEQ ID NO: 3 - DNA, 24 bp, Artificial Sequence
  Sonde
tagttgtgatgcwatcatgactag